(19) United States
(12) Patent Application Publication
(10) Pub. No.: US 2015/0216214 A1
(43) Pub. Date: Aug. 6, 2015

(54) FOOD COMPOSITION CONTAINING FRESH SPIRULINA AND PRODUCTION METHOD THEREOF

(71) Applicant: Eric Dareville, Meursanges (FR)

(72) Inventor: Eric Dareville, Meursanges (FR)

(21) Appl. No.: 14/424,178

(22) PCT Filed: Aug. 26, 2013
(86) PCT No.: PCT/FR2013/051965

§ 371 Date: Feb. 26, 2015

(30) Foreign Application Priority Data
Aug. 27, 2012 (FR) 1258003

(51) Int. Cl. A23L 1/30; A61K 45/06

(52) U.S. Cl. CPC A23L 1/3014; A61K 45/06

(57) ABSTRACT

The food composition containing fresh spirulina, includes: at least 50 mass-% fresh spirulina having a water content greater than or equal to 15%; 0.1 to 3% antioxidant agents and antimicrobial agents extracted from plants; and 0.1 to 20% fatty food substance. A method for the production of such composition is also described.

### TECHNICAL FIELD

The subject of the present invention is a food composition based on fresh spirulina and also a process for preparing such a composition.

### PRIOR ART

The term “spirulina” covers herein the edible species of cyanobacteria of the Arthrospira genus and especially in particular the species Arthrospira platensis or maxima.

Spirulina is a product that is very advantageous from a nutritional point of view. This is because it contains 55% to 65% of vegetable proteins. It also contains essential fatty acids: omega 6, gamma-linolenic acid. Spirulina also contains vitamins: A, E, D, B1, B2, B3, B6, B7, B8, K and beta-carotene. Spirulina also consists of minerals and trace elements: calcium, phosphorus, magnesium, iron, zinc, copper, manganese, chromium, sodium, potassium and selenium. In addition, spirulina is very rich in chlorophyll and in phycocyanin, which are pigments with notable detoxifying, antioxidant and free-radical-scavenging properties. Finally, enzymes are contained in spirulina, the most important of which is superoxide dismutase (SOD) which contains iron.

Spirulina is therefore a very effective food supplement, a few grams consumed daily making it possible to overcome a number of nutritional deficiencies.

Furthermore, the culturing of spirulina is very advantageous from a productivity and ecology point of view: a low consumption of water compared with other sources of vegetable or animal proteins and very high yields per hectare (50 t/ha). In addition, no phyto-sanitary, pesticidal or antibiotic product is necessary.

However, its consumption and its marketing in fresh form is not widespread owing to the fact that it rapidly rots shortly after harvesting. Thus, virtually all the marketed spirulina is in dried/dehydrated form, as dried twigs or as powder. However, in this form, spirulina has a pronounced taste which can curb its consumption. Furthermore, the dehydration is accompanied by a decrease in the nutritional qualities.

The present invention therefore aims to provide a novel form of preparation of spirulina which makes it possible to preserve over time the nutritional and organoleptic characteristics of the fresh spirulina thus prepared, and which is also economical to implement and applicable on a large scale.

### SUMMARY OF THE INVENTION

The present invention relates to a food composition based on fresh spirulina. This composition comprises:
at least 50% by mass of fresh spirulina with a water content greater than or equal to 15%;
0.1% to 4% of antioxidant and antimicrobial agents extracted from plants;
0.1% to 20% of dietary fatty substance.

In the present text, the term “based on” is intended to mean that the fresh spirulina is the principal and major ingredient of the composition.

The invention is also related to a process for preparing a food composition based on fresh spirulina as previously described.

This process comprises the following steps:
harvesting the spirulina;
filtering and pressing;
transferring into a mixing chamber and placing under vacuum an amount of filtered and pressed fresh spirulina such that it will represent at least 50% of the final total mass;
introducing antioxidant and antimicrobial agents extracted from plants into the chamber under vacuum, followed by mixing;
introducing a dietary fatty substance into the chamber under vacuum, followed by mixing;
breaking the vacuum, recovering the composition and packaging.

It is clearly understood that the process for preparing the composition prevents oxidation of the mass of harvested and pressed fresh spirulina, making it possible to store it, once packaged, for several months at ambient temperature. In addition, it advantageously proves to be the case that the taste of the spirulina thus prepared is less marked than that of the dehydrated preparations and thus facilitates its consumption.

Other advantages and characteristics of the invention will emerge more clearly from the detailed description which follows.

### DETAILED DESCRIPTION OF THE INVENTION

A first subject of the invention is therefore a food composition based on fresh spirulina. This composition comprises:
at least 50% by mass of fresh spirulina with a water content greater than or equal to 15%;
0.1% to 4% of antioxidant and antimicrobial agents extracted from plants;
0.1% to 20% of dietary fatty substance.

The term “fresh spirulina” is intended to mean a non-dried, non-frozen spirulina harvested less than 12 h previously; according to the preparation process, this fresh spirulina is filtered and pressed and comprises a water content strictly greater than 10%, preferably greater than or equal to 15%.

The composition may also comprise another vegetable or animal ingredient, depending on the desired taste and the desired texture: the total principal ingredients reaches 75% to 99% by weight, made up to 100% with oil or fatty substance and the plant extracts having antioxidant and/or antimicrobial properties.

Preferably, the composition comprises at least 80% of fresh spirulina. Most preferably it contains at least 85% of spirulina.

According to a first characteristic, the plant extracts performing the function of an antioxidant agent are taken from the group consisting of: rosemary extract, sage extract, fenugreek extract, holy basil extract, red clover extract, galanga extract, ginger extract, ginseng extract, tomato lycopene, curcumin from Curcuma longa, ascorbic acid from acerola or other fruits. Preferably, the extract is a rosemary extract.

According to another characteristic, the plant extracts performing the function of an antimicrobial agent are taken from the group consisting of: extracts and/or essential oils of oregano (Origanum vulgare, Origanum compactum), from common thymes (Thymus vulgaris), of marjoram (Origanum majorana), of cinnamon leaf or bark (Cinnamomum zeylanicum or Cinnamomum verum), of clove (Syzygium aromaticum), of coriander (Coriandrum sativum), of garlic, oleoresinous extracts of Capsicum, old man's beard (Usnea barbata), pepper extract, essential oil of tea tree (Melaleuca alternifolia), spices, mustard and wasabi. In particular, the antimicrobial agents are sulfur-containing allicin derivatives extracted from plants of the Allium genus, and more precisely from the Allium sativum and Allium cepa varieties. Preferably, the extracts are oregano, thyme, marjoram, cinnamon leaf, clove and coriander extracts.

The plant extracts mentioned above can also be super-critical carbon dioxide extracts.

According to a final characteristic, the dietary fatty substance is preferably a food oil. It will also be possible to use a single oil or a mixture of different oils and, where appropriate, of animal fats (butter, lard) without departing from the context of the present invention. Preferably, a food oil rich in omega 3 will be chosen: this advantageously makes it possible to confer a more equilibrated omega 3/omega 6 ratio on the food composition, owing to the omega 6 content of spirulina. Thus, the food oil is preferably taken, alone or in combination, from the group consisting of chia oil, linseed oil, camelina oil, rapeseed oil, fish oil or any other sources of food oils or dietary fatty substances rich in omega 3. The oil is preferably chia oil.

Preferably, the proportion of fatty substance, and in particular of oil, is less than or equal to 5% by weight.

The composition according to the invention may also comprise vitamin C as oxygen scavenger and tocopherol-regenerating agent.

The invention also relates to a process for preparing a food composition based on fresh spirulina as previously described. This process comprises the following steps:
harvesting the spirulina;
filtering and pressing; the filtering is carried out on microporous filters (30 microns);
transferring into a mixing chamber and placing under vacuum an amount of filtered and pressed fresh spirulina such that it will represent at least 50% of the final total mass;
introducing antioxidant and antimicrobial agents as previously defined into the chamber under vacuum, followed by mixing;
introducing food oil into the chamber under vacuum, followed by mixing;
breaking the vacuum, recovering the composition and packaging.

Placing the mixing chamber under vacuum makes it possible to eliminate the air in contact with the mass of spirulina and to prevent oxidation thereof. The vacuum may reach −96 kPa.

This placing under vacuum results in partial dehydration of spirulina, without however falling below a water content of less than 15%.

According to one embodiment, said chamber may be connected to a second chamber containing zeolites, such that the atmosphere of the first chamber circulates inside the second chamber. The partial dehydration conditions and the removal of the surface water are thus improved. However, it will be noted that the residence time of the ingredients in the chamber is short, a few minutes, at most a few tens of minutes.

In addition, an optional step of rinsing with drinking water before pressing may be carried out. It makes it possible to remove a part of the salt originating from the spirulina culture medium and can be recycled in a closed circuit by means of an ultrafiltration, a decontamination by UV radiation and a further calibration.

Preferentially, said rinsing is carried out with sterile water. Even more preferentially, the pH, salinity and alkalinity parameters of said sterile water are controlled.

According to one preferred embodiment, the packaging is carried out under vacuum, for example by vapor jet for capped glass jars or with a vacuum packaging machine for weldable bags, optionally under a protective atmosphere of neutral gas, of the CO₂ or nitrogen type.

According to another advantageous embodiment, the packaging uses pascalization, i.e. a very high pressure treatment. Recourse to this treatment advantageously makes it possible to drastically reduce the content of fatty substance and of antimicrobial and antioxidant agents present in the composition, to less than 1% by weight each, up to 0.1%.

A food preparation based on fresh spirulina is thus obtained, which can be stored for at least 12 months at ambient temperature, without detrimental modification of the organoleptic qualities of the composition.

### INDUSTRIAL APPLICATION

The composition and the process for obtaining same can be used in food-processing facilities in the immediate proximity of spirulina production and harvesting areas.

1. A food composition based on fresh spirulina, comprising:
at least 50% by mass of fresh spirulina with a water content greater than or equal to 15%;
0.1% to 4% of antioxidants and antimicrobial agents extracted from plants; and
0.1% to 20% of dietary fatty substance.

2. The composition as claimed in claim 1, wherein said composition comprises at least 80% of fresh spirulina.

3. The composition as claimed in claim 1, wherein the plant extracts performing the function of an antioxidant are selected from the group consisting of: rosemary extract, sage extract, fenugreek extract, holy basil extract, red clover extract, galanga extract, ginger extract, ginseng extract and tomato lycopene.

4. The composition as claimed in claim 1, wherein the plant extracts performing the function of an antimicrobial agent are selected from the group consisting of: extracts and/or essential oils of oregano (Origanum vulgare, Origanum compactum), from common thymes (Thymus vulgaris), of marjoram (Origanum majorana), of cinnamon leaf or bark (Cinnamomum zeylanicum or Cinnamomum verum), of clove (Syzygium aromaticum), of coriander (Coriandrum sativum), of garlic, oleoresinous extracts of Capsicum, old man's beard (Usnea barbata), pepper extract, essential oil of tea tree (Melaleuca alternifolia), spices, mustard and wasabi.

5. The composition as claimed in claim 1, wherein the dietary fatty substance is a food oil taken, alone or in combination, from: chia oil, linseed oil, camelina oil, rapeseed oil, fish oil or any other sources of food oils or dietary fatty substances.

6. A process for preparing a food composition based on fresh spirulina as claimed in claim 1, which comprises the following steps:
harvesting the spirulina;
filtering and pressing;
transferring into a mixing chamber and placing under vacuum an amount of filtered and pressed spirulina such that it will represent at least 50% of the final total mass;
introducing antioxidants selected from the group consisting of rosemary extract, sage extract, fenugreek extract, holy basil extract, red clover extract, galanga extract, ginger extract, ginseng extract and tomato lycopene and antimicrobial agents selected from the group consisting of: extracts and/or essential oils of oregano (Origanum vulgare, Origanum compactum), from common thymes (Thymus vulgaris), of marjoram (Origanum majorana), of cinnamon leaf or bark (Cinnamomum zeylanicum or Cinnamomum verum), of clove (Syzygium aromaticum), of coriander (Coriandrum sativum), of garlic, oleoresinous extracts of Capsicum, old man's beard (Usnea barbata), pepper extract, essential oil of tea tree (Melaleuca alternifolia), spices, mustard and wasabi into the chamber under vacuum, followed by mixing;
introducing a food oil into the chamber under vacuum, followed by mixing; and
breaking the vacuum, recovering the composition and packaging.

7. The process for preparing a food composition based on fresh spirulina as claimed in claim 6, further comprising a step of rinsing with drinking water before the pressing.

8. The process for preparing a food composition based on fresh spirulina as claimed in claim 6, wherein the packaging is under vacuum.

9. The process for preparing a food composition based on fresh spirulina as claimed in claim 6, wherein the packaging uses pascalization.

10. The composition as claimed in claim 2, wherein the plant extracts performing the function of an antioxidant are selected from the group consisting of: rosemary extract, sage extract, fenugreek extract, holy basil extract, red clover extract, galanga extract, ginger extract, ginseng extract and tomato lycopene.

11. The composition as claimed in claim 2, wherein the plant extracts performing the function of an antimicrobial agent are selected from the group consisting of: extracts and/or essential oils of oregano (Origanum vulgare, Origanum compactum), from common thymes (Thymus vulgaris), of marjoram (Origanum majorana), of cinnamon leaf or bark (Cinnamomum zeylanicum or Cinnamomum verum), of clove (Syzygium aromaticum), of coriander (Coriandrum sativum), of garlic, oleoresinous extracts of Capsicum, old man's beard (Usnea barbata), pepper extract, essential oil of tea tree (Melaleuca alternifolia), spices, mustard and wasabi.

12. The composition as claimed in claim 3, wherein the plant extracts performing the function of an antimicrobial agent are selected from the group consisting of: extracts and/or essential oils of oregano (Origanum vulgare, Origanum compactum), from common thymes (Thymus vulgaris), of marjoram (Origanum majorana), of cinnamon leaf or bark (Cinnamomum zeylanicum or Cinnamomum verum), of clove (Syzygium aromaticum), of coriander (Coriandrum sativum), of garlic, oleoresinous extracts of Capsicum, old man's beard (Usnea barbata), pepper extract, essential oil of tea tree (Melaleuca alternifolia), spices, mustard and wasabi.

13. The composition as claimed in claim 2, wherein the dietary fatty substance is a food oil taken, alone or in combination, from: chia oil, linseed oil, camelina oil, rapeseed oil, fish oil or any other sources of food oils or dietary fatty substances.

14. The composition as claimed in claim 3, wherein the dietary fatty substance is a food oil taken, alone or in combination, from: chia oil, linseed oil, camelina oil, rapeseed oil, fish oil or any other sources of food oils or dietary fatty substances.

15. The composition as claimed in claim 4, wherein the dietary fatty substance is a food oil taken, alone or in combination, from: chia oil, linseed oil, camelina oil, rapeseed oil, fish oil or any other sources of food oils or dietary fatty substances.

16. The composition as claimed in claim 12, wherein the dietary fatty substance is a food oil taken, alone or in combination, from: chia oil, linseed oil, camelina oil, rapeseed oil, fish oil or any other sources of food oils or dietary fatty substances.

17. The process for preparing a food composition based on fresh spirulina as claimed in claim 7, wherein the packaging is under vacuum.
